(19) United States
(12) Patent Application Publication
(10) Pub. No.: US 2025/0259700 A1
(43) Pub. Date: Aug. 14, 2025

(54) PROBABILISTIC IDENTIFICATION OF FEATURES FOR MACHINE LEARNING ENABLED CELLULAR PHENOTYPING

(71) Applicant: Genentech, Inc., South San Francisco, CA (US)

(72) Inventors: Lisa Michelle MCGINNIS, South San Francisco, CA (US); Tatiana NOVITSKAYA, Palo Alto, CA (US); Andries ZIJLSTRA, Alameda, CA (US)

(21) Appl. No.: 19/196,053

(22) Filed: May 1, 2025

Related U.S. Application Data
(63) Continuation of application No. PCT/US2023/036521, filed on Oct. 31, 2023.
(60) Provisional application No. 63/382,075, filed on Nov. 2, 2022.

(51) Int. Cl. G16B 20/00; G06V 10/40; G06V 20/69; G16B 40/00

(52) U.S. Cl. CPC G16B 20/00; G06V 10/40; G06V 20/69; G16B 40/00

(57) ABSTRACT

A method may include extracting a plurality of features for each cell depicted in an image. A biomarker identification model may be applied to determine, based on the features associated with each cell, whether the cell is associated with various biomarkers. A set of probabilities for each cell in the population of cells may be determined based on an output of the biomarker identification model. The set of probabilities may include, for each biomarker, a probability of a corresponding cell being associated with the biomarker. One or more subsets of cells, each of which corresponding to a different cellular phenotype, may be identified based on the set of probabilities associated with each cell. A feature set associated with each subset of cells may be identified as being indicative of a probability of a cell being associated with a corresponding phenotype. Related systems and computer program products are also provided.

### CROSS-REFERENCE TO RELATED APPLICATIONS

This application is a continuation of U.S. Patent Application No. PCT/US2023/036521, filed on Oct. 31, 2023, which claims priority to U.S. Provisional Patent Application No. 63/382,075, entitled “Probabilistic Identification of Features for Machine Learning Enabled Cellular Phenotyping,” filed Nov. 2, 2022, the disclosure of which is incorporated herein by reference in its entirety.

### TECHNICAL FIELD

The subject matter described herein relates generally to the digital and computational pathology and more specifically to a probabilistic approach to identifying features for machine learning enabled determination of cellular phenotypes.

### BACKGROUND

A cell's phenotype may refer to a unique combination of morphological and functional characteristics that result from various cellular processes including, for example, gene expression, protein expression, and/or the like. In some cases, complex interactions between a cell's genome, epigenome, and local environment may give rise to an assortment of observable characteristics collectively known as the cell's phenotype. While cellular phenotypes, including the phenotypes of tumor cells, are typically attributed to genomic instability, increasing attention has recently been given to epigenetic and microenvironmental influences. Such non-genetic factors can further increase the intrinsic diversity and plasticity of tumor cells. At the tumor level, non-genetic factors can contribute to greater phenotypic heterogeneity that allows tumor cells to evade immune responses and resist drug intervention.

### SUMMARY

Systems, methods, and articles of manufacture, including computer program products, are provided for probabilistic identification of features for machine learning enabled cellular phenotyping. In one aspect, there is provided a system for probabilistic identification of features for machine learning enabled cellular phenotyping. The system may include at least one processor and at least one memory. The at least one memory may include program code that provides operations when executed by the at least one processor. The operations may include: extracting, from a first image depicting a population of cells, a plurality of features for each cell in the population of cells; applying a biomarker identification model to determine, based at least on the plurality of features associated with each cell in the population of cells, whether the cell is associated with, positive for, or negative for a plurality of biomarkers; determining, based at least on an output of the biomarker identification model, a set of probabilities for each cell in the population of cells, and the set of probabilities including, for each biomarker in the plurality of biomarkers, a probability of a corresponding cell being associated with, positive for, or negative for the biomarker; identifying, based at least on the set of probabilities associated with each cell in the population of cells, a first subset of cells exhibiting a first phenotype; and identifying a first feature set associated with the first subset of cells as being indicative of a first probability of a cell being associated with, positive for, or negative for the first phenotype.

In another aspect, there is provided a method for probabilistic identification of features for machine learning enabled cellular phenotyping. The method may include: extracting, from a first image depicting a population of cells, a plurality of features for each cell in the population of cells; applying a biomarker identification model to determine, based at least on the plurality of features associated with each cell in the population of cells, whether the cell is associated with, positive for, or negative for a plurality of biomarkers; determining, based at least on an output of the biomarker identification model, a set of probabilities for each cell in the population of cells, and the set of probabilities including, for each biomarker in the plurality of biomarkers, a probability of a corresponding cell being associated with, positive for, or negative for the biomarker; identifying, based at least on the set of probabilities associated with each cell in the population of cells, a first subset of cells exhibiting a first phenotype; and identifying a first feature set associated with the first subset of cells as being indicative of a first probability of a cell being associated with, positive for, or negative for the first phenotype.

In another aspect, there is provided a computer program product for probabilistic identification of features for machine learning enabled cellular phenotyping. The computer program product may include a non-transitory computer readable medium storing instructions that cause operations when executed by at least one data processor. The operations may include: extracting, from a first image depicting a population of cells, a plurality of features for each cell in the population of cells; applying a biomarker identification model to determine, based at least on the plurality of features associated with each cell in the population of cells, whether the cell is associated with, positive for, or negative for a plurality of biomarkers; determining, based at least on an output of the biomarker identification model, a set of probabilities for each cell in the population of cells, and the set of probabilities including, for each biomarker in the plurality of biomarkers, a probability of a corresponding cell being associated with, positive for, or negative for the biomarker; identifying, based at least on the set of probabilities associated with each cell in the population of cells, a first subset of cells exhibiting a first phenotype; and identifying a first feature set associated with the first subset of cells as being indicative of a first probability of a cell being associated with, positive for, or negative for the first phenotype.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to the identification of features for machine learning enabled cellular phenotyping, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a digital pathology system, in accordance with some example embodiments;

FIG. 2 depicts a flowchart illustrating an example of a process for probabilistic feature identification for machine learning enabled cellular phenotyping, in accordance with some example embodiments;

FIG. 3 depicts a schematic diagram illustrating an example of a workflow for probabilistic identification of features for machine learning enabled cellular phenotyping, in accordance with some example embodiments;

FIG. 4 depicts examples of features extracted from an image depicting a population of cells, in accordance with some example embodiments;

FIG. 5A depicts a schematic diagram illustrating an example of a process for training and validating a biomarker identification model, in accordance with some example embodiments;

FIG. 5B depicts a schematic diagram illustrating an example of a process for training and validating a phenotype identification model, in accordance with some example embodiments;

FIG. 6A depicts a visualization of an example of a reduced dimension representation of a biomarker probabilities dataset, in accordance with some example embodiments;

FIG. 6B depicts another a visualization of an example of a reduced dimension representation of a biomarker probabilities dataset, in accordance with some example embodiments; and

FIG. 7 depicts a block diagram illustrating an example of a computing system, in accordance with some example embodiments.

When practical, similar reference numbers denote similar structures, features, or elements.

### DETAILED DESCRIPTION

In highly heterogeneous diseases such as cancer, insights into the phenotypes of cells forming diseased tissue and the surrounding microenvironment may be integral to the accurate diagnosis of disease subtype, prognosis of disease progress, and prediction of response to various treatments. For example, non-Hodgkin's lymphoma patients at high risk of disease progression with standard of care treatment (e.g., combination immunochemotherapy R-CHOP (rituximab, cyclophosphamide, doxorubicin, vincristine, and prednisone)) may be identified by characterizing the immune microenvironment, which includes identifying lymph node resident immune cells and infiltrating neoplastic cells. Nevertheless, conventional histological analysis techniques for identifying the phenotypes of cells depicted in a microscopic image (e.g., a hematoxylin and cosin (H&E) stained whole slide image, a multiplex immunofluorescence (MxIF) stained whole slide image, and/or the like) tend to be error prone due to a high level of inter- and intra-pathologist variability. Meanwhile, existing machine learning based solutions are also susceptible to inter- and intra-pathologist variability at least because the training of machine learning based cellular phenotyping models relies on expert annotation of training samples but expert annotations are not sufficiently reliable due to the presence of inter- and intra-pathologist variability.

In some example embodiments, a machine learning based phenotype identification model may be trained to determine, based on one or more features extracted from an image depicting a population of cells, the probability that one or more of the cells depicted in the image are associated with, positive for, or negative for a particular phenotype. The machine learning based phenotype identification model may be trained to generate a probabilistic output instead of a binary output in order to provide a more precise quantification of the error (or uncertainty) present in the determination that the one or more cells depicted in the image are positive (or negative) for a particular phenotype. In some cases, one or more downstream tasks, such as the determination of a disease diagnosis, a disease progression, a disease burden, and/or a treatment response, may be performed when the probability that one or more of the cells depicted in the image are positive for a particular phenotype satisfies one or more thresholds. Moreover, in some cases, the same machine learning based phenotype identification model or one or more separate machine learning based phenotype identification models may be trained to determine the probability that one or more of the cells depicted in the image are positive for another phenotype.

In some example embodiments, one or more features present in an image may be identified as being indicative of a cell being associated with, positive for, or negative for a particular phenotype or a probability of the cell being associated with, positive for, or negative for the particular phenotype. For example, in some cases, a plurality of features may be extracted from an image depicting a population of cells including, for example, a hematoxylin and cosin (H&E) stained whole slide image, a multiplex immunofluorescence (MxIF) stained whole slide image, and/or the like. These features may be collected over multiple channels. For instance, in some cases, each channel may correspond to one or more of an emission wavelength of a fluorescent dye applied to the image, a metal ion collected by a mass cytometer, a nucleotide sequence identified by barcode hybridization, a nucleotide sequence identified by sequencing, and/or the like. The one or more features that are indicative of a cell being associated with, positive for, or negative for a particular phenotype or the probability of the cell being associated with, positive for, or negative for the particular phenotype may include a combination of features that differentiate one subset of cells from another subset of cells depicted in the image.

In some example embodiments, one or more subsets of cells present in the image may be identified by applying a biomarker identification model including, for example, a machine learning based biomarker identification model. For example, in some cases, the biomarker identification model may be applied to determine, based at least on the features associated with each cell in the population of cells depicted in the image, whether the cell is associated with, positive for, or negative for a plurality of biomarkers. The output of the biomarker identification model may include a set of probabilities, each of which being a probability of the cell being associated with, positive for, or negative for a corresponding biomarker. The biomarker identification model may generate a probabilistic output instead of a binary output in order to provide a more precise quantification of the error (or uncertainty) present in the determination that the individual cells depicted in the image are positive (or negative) for a particular biomarker. For example, a binary output may include either a first value (e.g., “1”) to indicate that a cell is positive for a biomarker or a second value (e.g., “0”) to indicate that the cell is negative for the biomarker even though there is uncertainty in whether the cell is positive (or negative) for the biomarker. A probabilistic output, such as a probability that the cell is positive (or negative) for the biomarker, may capture the uncertainty that is included in the determination that the cell is positive (or negative) for the biomarker.

In some example embodiments, the one or more subsets of cells may be identified based at least on the set of probabilities associated with each cell depicted in the image. For example, in some cases, each subset of cells may correspond to one or more clusters of cells present in a reduced dimension representation of a dataset including the set of probabilities associated with each cell. The features that are associated with each subset of cells may be identified as being indicative of a cell being associated with, positive for, or negative for a corresponding phenotype or a probability of the cell being associated with, positive for, or negative for the corresponding phenotype. For instance, a first feature set associated with a first subset of cells may be identified as being indicative of a cell being associated with, positive for, or negative for a first phenotype (or a probability of the cell being associated with, positive for, or negative for the first phenotype) while a second feature set associated with a second subset of cells may be identified as being indicative of the cell being positive being a second phenotype (or a probability of the cell being associated with, positive for, or negative for the second phenotype. In some cases, a first phenotype identification model may be trained to determine a first probability of a cell being associated with, positive for, or negative for the first phenotype based on the first feature set associated with the first subset of cells while a second phenotype identification model may be trained to determine a second probability of a cell being associated with, positive for, or negative for the second phenotype based on the second feature set associated with the second subset of cells.

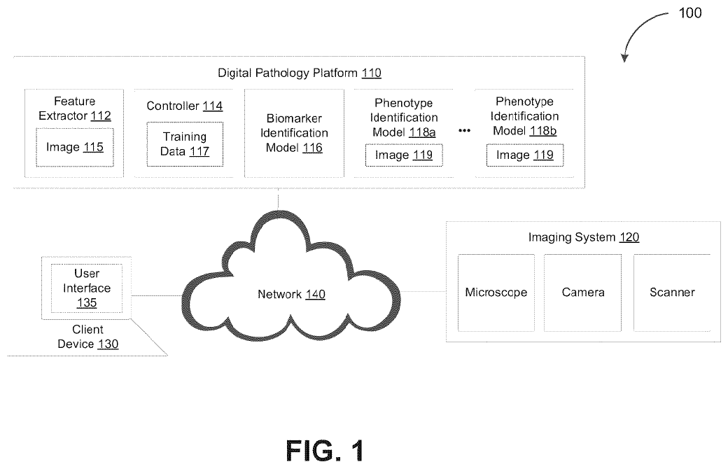

FIG. 1 depicts a system diagram illustrating an example of a digital pathology system 100, in accordance with some example embodiments. Referring to FIG. 1, the digital pathology system 100 may include a digital pathology platform 110, an imaging system 120, and a client device 130. As shown in FIG. 1, the digital pathology platform 110, the imaging system 120, and the client device 130 may be communicatively coupled via a network 140. The network 140 may be a wired network and/or a wireless network including, for example, a local area network (LAN), a virtual local area network (VLAN), a wide area network (WAN), a public land mobile network (PLMN), the Internet, and/or the like. The imaging system 120 may include one or more imaging devices including, for example, a microscope, a digital camera, a whole slide scanner, a robotic microscope, and/or the like. The client device 130 may be a processor-based device including, for example, a workstation, a desktop computer, a laptop computer, a smartphone, a tablet computer, a wearable apparatus, and/or the like.

Referring again to FIG. 1, the digital pathology platform 110 may include a feature extractor 112, a controller 114, a biomarker identification model 116, and one or more phenotype identification models 118. As shown in FIG. 1, the feature extractor 112 may extract, from a first image 115 depicting a population of cells, a plurality of features associated with each cell in the population of cells. In some cases, the first image 115 may be a stained whole slide image (WSI) including, for example, a hematoxylin and cosin (H&E) stained whole slide image, a multiplex immunofluorescence (MxIF) stained whole slide image, and/or the like. Moreover, in some cases, the feature extractor 112 may collect, for each cell in the population of cells depicted in the first image 115, the plurality of features over multiple channels. For example, in some instances, each channel (e.g., each individual feature) may correspond to one or more of an emission wavelength of a fluorescent dye applied to the first image 115. Alternatively and/or additionally, each channel (e.g., each individual feature) may correspond to a metal ion collected by a mass cytometer, a nucleotide sequence identified by barcode hybridization, a nucleotide sequence identified by sequencing, and/or the like.

In some example embodiments, the controller 114 may apply the biomarker identification model 116 to determine, based at least on the features associated with each cell depicted in the first image 115, whether the cell is associated with, positive for, or negative for each biomarker of a plurality of biomarkers. Examples of biomarkers may include Pax5, CD68, CD3, CD8, Foxp3, CD335, and Ki67. In some cases, the biomarker identification model 116 may implemented using one or more machine learning models including, for example, a gradient boosted decision tree, a random forest, a naïve Bayes classifier, a neural network, a k-means clustering model, a logistic regression model, and/or the like. Moreover, the output of the biomarker identification model 116 may include, for each cell of the population of cells depicted in the first image 115, a set of probabilities, each of which being a probability that the cell is associated with, positive for, or negative for a corresponding biomarker. For example, for a set of n biomarkers b₁, b₂, . . . , bₙ, the output of the biomarker identification model 116 may include, for each cell in the population of cells depicted in the first image 115, a set of n probabilities P(b₁), P(b₂), . . . , P(bₙ). That is, the output of the biomarker identification model 116 may include, for each cell in the population of cells depicted in the first image 115, a set of probabilities that include a first probability of the cell being associated with, positive for, or negative for a first biomarker and a second probability of the cell being associated with, positive for, or negative for a second biomarker.

In some example embodiments, the controller 114 may identify one or more features that are indicative of a cell being associated with, positive for, or negative for a particular phenotype or a probability of the cell being associated with, positive for, or negative for the particular phenotype. For example, in some cases, the controller 114 may identify, based at least on the set of probabilities associated with each cell depicted in the first image 115, one or more subsets of cells present in the population of cells depicted in the first image 115. Each of the subsets of cells identified within the population of cells depicted in the first image 115 may correspond to a separate phenotype. Moreover, the feature set that is associated with each subset of cells identified within the population of cells depicted in the first image 115 may be indicative of whether a cell is associated with, positive for, or negative for a corresponding phenotype or a probability of the cell being associated with, positive for, or negative for the corresponding phenotype.

In some example embodiments, the controller 114 may identify the one or more subsets of cells present in the population of cells depicted in the first image 115 by at least generating a reduced dimension representation of a dataset including the set of probabilities associated with each cell in the population of cells. For example, in some cases, the controller 114 may generate the reduced dimension representation of the dataset by applying t-distributed stochastic neighbor embedding (t-SNE), uniform manifold approximation and projection (UMAP), principal component analysis (PCA), linear discriminant analysis (LDA), a machine learning model, and/or the like. In some cases, the reduced dimension representation of the dataset may occupy a lower-dimensional space (e.g., a space defined by fewer features) than the dataset itself. For instance, the dataset including the set of probabilities associated with each cell in the population of cells may occupy an n-dimensional space in which each dimension (or feature) corresponds to a probability of the cell being associated with, positive for, or negative for one of the n-quantity of biomarkers. The reduced dimension representation of the dataset may occupy an m-dimensional space where m<n (or m<<n).

In some example embodiments, the reduced dimension representation of the dataset may occupy a two-dimensional (or three-dimensional) space that provides a visualization of the nexus between individual cells depicted in the first image 115. For example, in the reduced dimension representation of the dataset, individual cells may be spatially distributed in accordance with their respective probabilities of being associated with, positive for, or negative for each biomarker of the n-quantity of biomarkers. Cells that exhibit similar probabilities of being associated with, positive for, or negative for a similar combination of biomarkers may be proximately located in the m-dimensional space occupied by the reduced dimensional representation of the dataset. For instance, in some cases, cells having similar probabilities of being associated with, positive for, or negative for a similar combination of biomarkers may form one or more cell clusters in the m-dimensional space occupied by the reduced dimensional representation of the dataset. The controller 114 may identify each subset of cells to include one or more of the cell clusters present in the m-dimensional space occupied by the reduced dimensional representation of the dataset.

In some example embodiments, each subset of cells identified within the population of cells depicted in the first image 115 may correspond to a particular phenotype. In some cases, the phenotype of a cell may correspond to a transient cell state exhibited by the cell. Alternatively and/or additionally, other examples of phenotypes may include tumor cell, macrophage, regulatory T-cell, CD8-positive T-cell, B-cell, and natural killer (NK) cell. The controller 114 may identify the feature set associated with each subset of cells as being indicative of a cell being associated with, positive for, or negative for a corresponding phenotype or a probability of the cell being associated with, positive for, or negative for the corresponding phenotype. For example, a first feature set associated with a first subset of cells may be identified as being indicative of a cell being associated with, positive for, or negative for a first phenotype (or a probability of the cell being associated with, positive for, or negative for the first phenotype) while a second feature set associated with a second subset of cells may be identified as being indicative of the cell being positive being a second phenotype (or a probability of the cell being associated with, positive for, or negative for the second phenotype. Moreover, in some cases, the controller 114 may generate, based least on the first feature set and the second feature set, training data 117 for training the one or more phenotype identification model 118.

In some example embodiments, the controller 114 may train the one or more phenotype identification models 118 to determine, based at least on the feature set associated with a phenotype, whether a cell depicted in an second image 119 is associated with, positive for, or negative for the phenotype. For example, the controller 114 may train a first phenotype identification model 118a to determine, based at least on the first feature set extracted from the second image 119, whether a cell depicted in the second image 119 is associated with, positive for, or negative for the first phenotype. Furthermore, in some cases, the controller 114 may train a second phenotype identification model 118b to determine, based at least on the second feature set extracted from the second image 119, whether the cell depicted in the second image 119 is associated with, positive for, or negative for the second phenotype.

In some cases, the one or more phenotype identification models 118 may be implemented as one or more machine learning models including, for example, a gradient boosted decision tree, a random forest, a naïve Bayes classifier, a neural network, a k-means clustering model, a logistic regression model, and/or the like. Moreover, although FIG. 1 depicts the first phenotype identification model 118a and the second phenotype identification model 118b being implemented using two separate machine learning models, in some cases, a single machine learning model may implement multiple of the one or more phenotype identification models 118 (e.g., the first phenotype identification model 118a as well as the second phenotype identification model 118b). Furthermore, in some cases, a single machine learning model may also implement the biomarker identification model 116 as well as the one or more phenotype identification models 118.

As noted, in some example embodiments, the output of the biomarker identification model 116 for each cell depicted in the first image 115 may include a set of probabilities such as, for example, a set of n probabilities P(b₁), P(b₂), . . . , P(bₙ) in which each probability P(bᵢ) is a probability of the cell being associated with, positive for, or negative for a corresponding biomarker bᵢ. The biomarker identification model 116 may be trained to generate a probabilistic output instead of a binary output in order to provide a more precise quantification of the error (or uncertainty) present in the determination that the individual cells depicted in the first image 115 are positive (or negative) for each biomarker bᵢ. In some cases, the output of the one or more phenotype identification models 118 may also include a probability of a cell being associated with, positive for, or negative for a corresponding phenotype. It should be appreciated that the one or more phenotype identification models 118 may also be trained to generate a probabilistic output instead of a binary output in order to provide a more precise quantification of the error (or uncertainty) present in the determination that a cell is positive (or negative) for a particular phenotype.

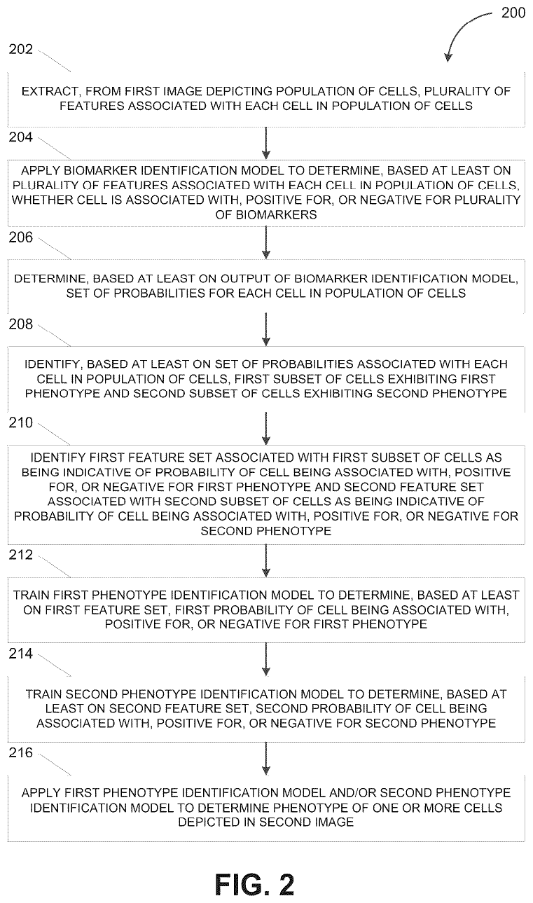

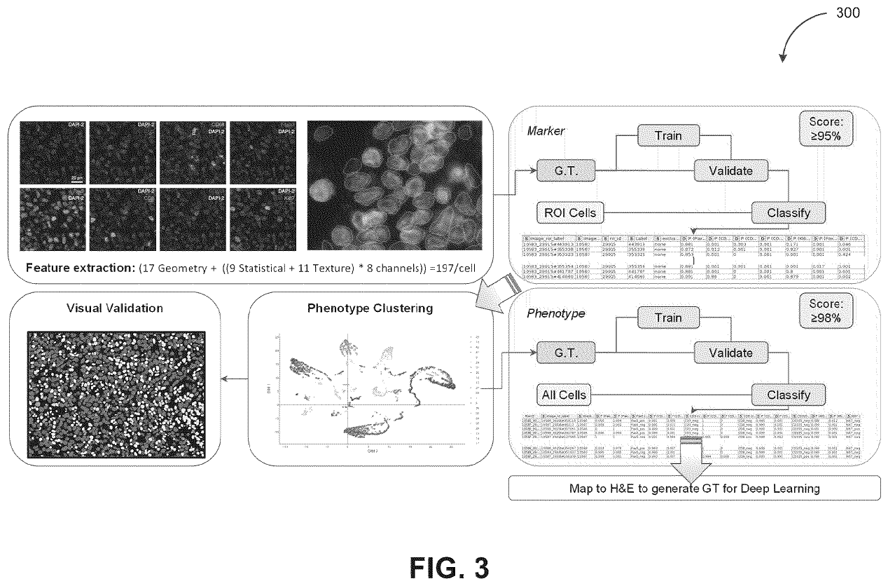

FIG. 2 depicts a flowchart illustrating an example of a process 200 for probabilistic feature identification for machine learning enabled cellular phenotyping, in accordance with some example embodiments. A corresponding workflow 300 for probabilistic feature identification for machine learning enabled cellular phenotyping is shown in FIG. 3. Referring to FIGS. 1-3, the process 200 (and the corresponding workflow 300) may be performed by the digital pathology platform 110 including, for example, by one or more of the feature extractor 112, the controller 114, the biomarker identification model 116, and the one or more phenotype identification models 118.

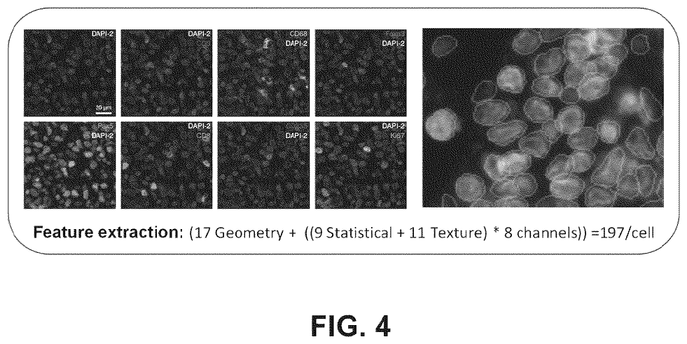

At 202, the feature extractor 112 may extract, from the first image 115 depicting a population of cells, a plurality of features for each cell in the population of cells. To further illustrate, FIG. 4 depicts examples of features extracted from the first image 115. As shown in FIG. 4, in some example embodiments, the feature extractor 112 may extract, from the first image 115, a plurality of features including, for example, one or more geometric features, statistical features, textural features, and/or the like. Moreover, FIG. 4 shows that the feature extractor 112 may extract the features over multiple channels. For example, in some cases, each channel (or feature) may correspond to one or more of an emission wavelength of a fluorescent dye applied to the image, a metal ion collected by a mass cytometer, a nucleotide sequence identified by barcode hybridization, a nucleotide sequence identified by sequencing, and/or the like. In the example shown in FIG. 4, a total of 197 features were extracted for each cell depicted in the first image 115. However, it should be appreciated that the feature extractor 112 may extract a different quantity of features from the first image 115 than the example shown in FIG. 4.

At 204, the controller 114 may apply the biomarker identification model 116 to determine, based at least on the plurality of features associated with each cell in the population of cells, whether the cell is associated with, positive for, or negative for a plurality of biomarkers. In some example embodiments, the controller 114 may apply the biomarker identification model 116 to determine, for each cell in the population of cells depicted in the first image 115, whether the cell is associated with, positive for, or negative for each of a plurality of biomarkers. For example, in some cases, the biomarker identification model 116 may be applied to determine, based on the features extracted from the first image 115, a probability that the individual cells depicted in the first image 115 are positive for the set of n biomarkers b₁, b₂, . . . , bₙ. As noted, examples of biomarkers may include Pax5, CD68, CD3, CD8, Foxp3, CD335, and Ki67.

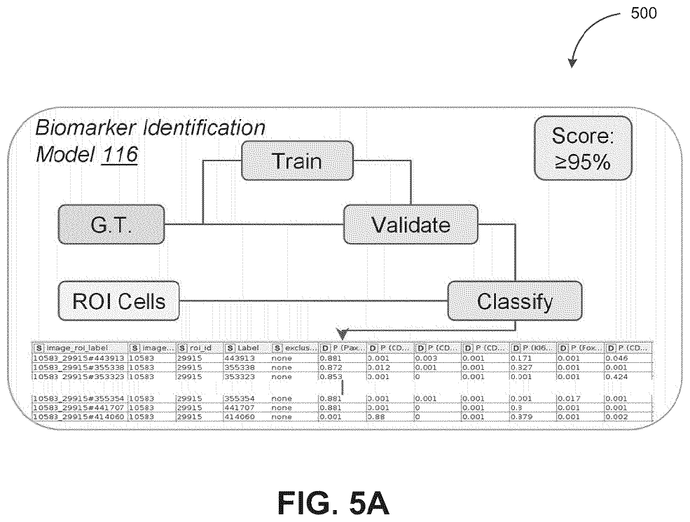

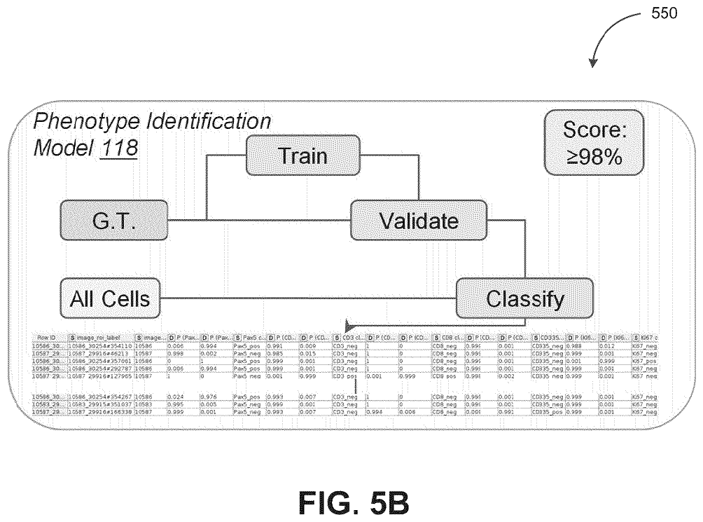

In some example embodiments, to train the biomarker identification model 116, the training data 117 may include, for each cell in the population of cells depicted in the first image 115, an annotated training sample including the plurality of features associated the cell and a ground truth label corresponding to each biomarker exhibited by the cell. In some cases, the ground truth labels assigned to a cell depicted in the first image 115 may be binary values such as, for example, a first value (e.g., 1) to indicate that the cell is associated with, positive for, or negative for a particular biomarker and a second value (e.g., 0) to indicate that the cell is negative for the biomarker. To further illustrate, FIG. 5A depicts a schematic diagram illustrating an example of a process 500 for training and validating the biomarker identification model 116, in accordance with some example embodiments. In the example shown in FIG. 5A, the biomarker identification model 116 may be trained to determine, for each region of interest (ROI) in the first image 115 corresponding to a cell, a set of a set of n probabilities P(b₁), P(b₂), . . . , P(bₙ), with each probability P(bᵢ) being a probability that the cell is associated with, positive for, or negative for the corresponding biomarker bᵢ.

At 206, the controller 114 may determine, based at least on an output of the biomarker identification model 116, a set of probabilities for each cell in the population of cells. In some example embodiments, the output of the biomarker identification model 116 may include, for each cell in the population of cells depicted in the first image 115, a set of n probabilities P(b₁), P(b₂), . . . , P(bₙ), with each probability P(bᵢ) being a probability that the cell is associated with, positive for, or negative for the corresponding biomarker bᵢ. The biomarker identification model 116 may be trained to generate a probabilistic output instead of a binary output in order to provide a more precise quantification of the error (or uncertainty) present in the determination that the individual cells depicted in the first image 115 are positive (or negative) for each biomarker bᵢ.

At 208, the controller 114 may identify, based at least on the set of probabilities associated with each cell in the population of cells, a first subset of cells exhibiting a first phenotype and a second subset of cells exhibiting a second phenotype. In some example embodiments, the controller 114 may identify the first subset of cells exhibiting the first phenotype and the second subset of cells exhibiting the second phenotype by at least generating a reduced dimension representation of a dataset including the set of n probabilities P(b₁), P(b₂), . . . , P(bₙ) associated with each cell in the population of cells depicted in the image 115. For example, in some cases, the controller 114 may generate the reduced dimension representation of the dataset by applying one or more of applying t-distributed stochastic neighbor embedding (t-SNE), uniform manifold approximation and projection (UMAP), principal component analysis (PCA), linear discriminant analysis (LDA), a machine learning model, and/or the like.

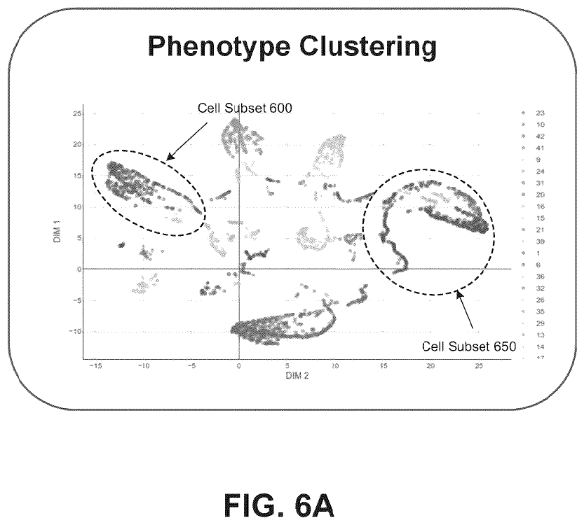

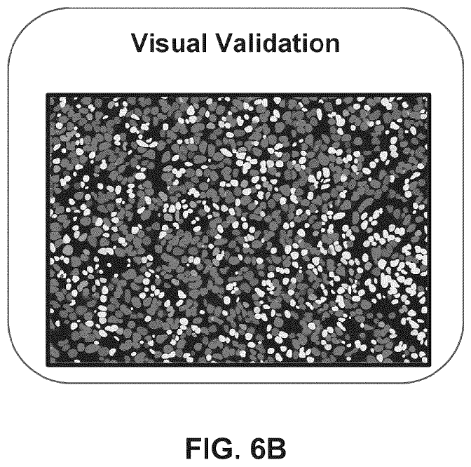

FIG. 6A depicts a visualization of an example of the reduced dimension representation of the dataset including the set of n probabilities P(b₁), P(b₂), . . . , P(bₙ) associated with each cell in the population of cells depicted in the image 115. While the dataset may occupy an n-dimensional space in which each dimension (or feature) corresponds to a probability of the cell being associated with, positive for, or negative for one of the n-quantity of biomarkers, the example of the reduced dimension representation of the dataset shown in FIG. 6A may occupy a two-dimensional space in which individual cells are spatially distributed in accordance with their respective probabilities of being associated with, positive for, or negative for each biomarker of the n-quantity of biomarkers. For example, the visualization of the reduced dimension representation of the dataset shown in FIG. 6A includes multiple clusters of cells with each cluster being populated by cells having similar probabilities of being associated with, positive for, or negative for a similar combination of biomarkers. FIG. 6B shows how the cells in each cell cluster are spatially distributed in the image 115. The controller 114 may thus identify, for example, a first cell subset 600 and a second cell subset 650, each of which containing one or more clusters of cells. As shown in FIG. 6B, some subsets of cells, such as the first cell subset 600, may include a single cluster of cells while some subsets of cells, such as the second cell subset 650, may include multiple clusters of cells. Moreover, in some cases, the controller 114 may identify the first cell subset 600 as being associated with a first phenotype and the second cell subset 650 as being associated with a second phenotype.

At 210, the controller 114 may identify a first feature set associated with the first subset of cells as being indicative of a first probability of a cell being associated with, positive for, or negative for the first phenotype and a second feature set associated with the second subset of cells as being indicative of a second probability of a cell being associated with, positive for, or negative for the second phenotype. For instance, in the example shown in FIGS. 6A-B, the first feature set associated with the first cell subset 600 may be identified as being indicative of a first probability of a cell being associated with, positive for, or negative for the first phenotype and the second feature set associated with the second cell subset 650 may be identified as being indicative of a second probability of a cell being associated with, positive for, or negative for the second phenotype.

At 212, the controller 114 may train the first phenotype identification model 118a to determine, based on the first feature set associated with the first subset of cells, the first probability of a cell being associated with, positive for, or negative for the first phenotype. To further illustrate, FIG. 5B depicts a schematic diagram illustrating an example of a process 550 for training and validating the one or more phenotype identification model 118. For example, in some cases, the controller 114 may train, based at least on the training data 117, the first phenotype identification model 118a to determine the first probability of a cell being associated with, positive for, or negative for the first phenotype. The training data 117 in this case may include, for each cell in the first cell subset 600, an annotated training sample including the first feature set associated with the first phenotype and a ground truth label corresponding to the first phenotype of the cell. For instance, in some cases, the ground truth label assigned to the cell may be a binary value such as a first value (e.g., 1) to indicate that the cell is associated with, positive for, or negative for the first phenotype and a second value (e.g., 0) to indicate that the cell is negative for the first phenotype. The first phenotype identification model 118a may thus be trained to learn a nexus between the first feature set and the first phenotype such that the first phenotype identification model 118a may determine the first probability of a cell being associated with, positive for, or negative for the first phenotype based on whether the cell exhibits one or more of the features included in the first feature set.

At 214, the controller 114 may train the second phenotype identification model 118b to determine, based at least on the second feature set associated with the second subset of cells, the second probability of a cell being associated with, positive for, or negative for the second phenotype. In some example embodiments, the one or more phenotype identification models 118 may be phenotype specific such that the controller 114 may train a separate phenotype identification model 118 for each possible phenotype. Accordingly, in some cases, the controller 114 may further train, based at least on the training data 117, the second phenotype identification model 118b to determine the second probability of a cell being associated with, positive for, or negative for the second phenotype. The training data 117 in this case may include, for each cell in the second cell subset 650, an annotated training sample including the second feature set associated with the second phenotype and a ground truth label corresponding to the second phenotype of the cell. In some cases, ground truth labels assigned to the cell may be a binary value such as, for example, a first value (e.g., 1) to indicate that the cell is associated with, positive for, or negative for second phenotype and a second value (e.g., 0) to indicate that the cell is negative for the second phenotype. Since the output of the When trained, the second phenotype identification model 118b may recognize the nexus between the second feature set and the second phenotype such that the second phenotype identification model 118b may determine the second probability of a cell being associated with, positive for, or negative for the second phenotype based on whether the cell exhibits one or more of the features included in the second feature set.

At 216, the controller 114 may apply the first phenotype identification model 118a and/or the second phenotype identification model 118b to determine a phenotype of one or more cells depicted in the second image 119. In some example embodiments, the controller 114 may apply the first phenotype identification model 118a to determine, based at least on the first feature set extracted from the second image 119, the first probability of one or more cells depicted in the second image 119 being associated with, positive for, or negative for the first phenotype. Furthermore, the controller 114 may apply the second phenotype identification model 118b to determine, based at least on the second feature set extracted from the second image 119, the second probability of one or more cells depicted in the second image 119 being associated with, positive for, or negative for the second phenotype.

In some example embodiments, one or more downstream tasks, such as a determination of a disease diagnosis, a disease progression, a disease burden, and/or a treatment response for a patient associated with the second image 119, may be performed based on the first probability of the one or more cells depicted in the second image 119 being associated with, positive for, or negative for the first phenotype and/or the second probability of the one or more cells depicted in the second image 119 being associated with, positive for, or negative for the second phenotype. Alternatively, in some cases, the one or more downstream tasks may be performed when the first probability of the one or more cells being associated with, positive for, or negative for the first phenotype and/or the second probability of the one or more cells being associated with, positive for, or negative for the second phenotype satisfy one or more thresholds. For example, in instances where the first probability of the one or more cells being associated with, positive for, or negative for the first phenotype and/or the second probability of the one or more cells being associated with, positive for, or negative for the second phenotype exceeds one or more thresholds, the presence (or absence) of cells having the first phenotype and/or the second phenotype may be used to determine a disease diagnosis, a disease progression, a disease burden, and/or a treatment response for the patient associated with the second image 119.

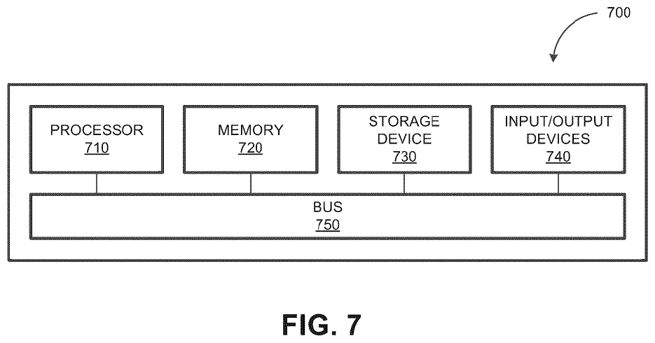

FIG. 7 depicts a block diagram illustrating an example of computing system 700, in accordance with some example embodiments. Referring to FIGS. 1 and 7, the computing system 700 may be used to implement the digital pathology platform 110, the imaging system 120, the client device 130, and/or any components therein.

As shown in FIG. 7, the computing system 700 can include a processor 710, a memory 720, a storage device 730, and an input/output device 740. The processor 710, the memory 720, the storage device 730, and the input/output device 740 can be interconnected via a system bus 750. The processor 710 is capable of processing instructions for execution within the computing system 700. Such executed instructions can implement one or more components of, for example, the digital pathology platform 110, the imaging system 120, the client device 130, and/or the like. In some example embodiments, the processor 710 can be a single-threaded processor. Alternately, the processor 710 can be a multi-threaded processor. The processor 710 is capable of processing instructions stored in the memory 720 and/or on the storage device 730 to display graphical information for a user interface provided via the input/output device 740.

The memory 720 is a computer readable medium such as volatile or non-volatile that stores information within the computing system 700. The memory 720 can store data structures representing configuration object databases, for example. The storage device 730 is capable of providing persistent storage for the computing system 700. The storage device 730 can be a floppy disk device, a hard disk device, an optical disk device, or a tape device, or other suitable persistent storage means. The input/output device 740 provides input/output operations for the computing system 700. In some example embodiments, the input/output device 740 includes a keyboard and/or pointing device. In various implementations, the input/output device 740 includes a display unit for displaying graphical user interfaces.

According to some example embodiments, the input/output device 740 can provide input/output operations for a network device. For example, the input/output device 740 can include Ethernet ports or other networking ports to communicate with one or more wired and/or wireless networks (e.g., a local area network (LAN), a wide area network (WAN), the Internet).

In some example embodiments, the computing system 700 can be used to execute various interactive computer software applications that can be used for organization, analysis and/or storage of data in various formats. Alternatively, the computing system 700 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 740. The user interface can be generated and presented to a user by the computing system 700 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

### Example Embodiments

Embodiments disclosed herein may include:
1. A computer-implemented method, comprising:
extracting, from a first image depicting a population of cells, a plurality of features for each cell in the population of cells;
applying a biomarker identification model to determine, based at least on the plurality of features associated with each cell in the population of cells, whether the cell is associated with, positive for, or negative for a plurality of biomarkers;
determining, based at least on an output of the biomarker identification model, a set of probabilities for each cell in the population of cells, and the set of probabilities including, for each biomarker in the plurality of biomarkers, a probability of a corresponding cell being associated with the biomarker;
identifying, based at least on the set of probabilities associated with each cell in the population of cells, a first subset of cells exhibiting a first phenotype; and
identifying a first feature set associated with the first subset of cells as being indicative of a first probability of a cell being associated with the first phenotype.
2. The method of embodiment 1, wherein the plurality of features include one or more geometric features, statistical features, and textural features.
3. The method of embodiment 1 or embodiment 2, wherein the plurality of features are collected over a plurality of channels, and wherein each channel of the plurality of channels corresponds to (i) an emission wavelength of a fluorescent dye applied to the first image, (ii) a metal ion collected by a mass cytometer, (iii) a nucleotide sequence identified by barcode hybridization, or (iv) a nucleotide sequence identified by sequencing.
4. The method of any one of embodiments 1-3, wherein each biomarker in the plurality of biomarkers corresponds to a protein of interest or an antigen comprising one or more carbohydrates, lipids, or nucleotides.
5. The method of any one of embodiments 1-4, wherein each biomarker in the plurality of biomarkers corresponds to a protein expressed by the population of cells.
6. The method of any one of embodiments 1-5, further comprising:
training a first phenotype identification model to determine, based on the first feature set associated with the first subset of cells, the first probability of the cell being associated with the first phenotype.
7. The method of embodiment 6, further comprising:
applying the first phenotype identification model to determine, based on the first feature set extracted from a second image, a probability of one or more cells depicted in the second image being associated with the first phenotype.
8. The method of embodiment 7, further comprising:
determining, based at least on the probability of the one or more cells depicted in the second image being associated with the first phenotype, a disease diagnosis, a disease progression, a disease burden, and/or a treatment response.
9. The method of embodiment 6, further comprising:
identifying, based at least on the set of probabilities associated with each cell in the population of cells, a second subset of cells exhibiting a second phenotype; and
training a second phenotype identification model to determine, based on a second feature set associated with the second subset of cells, a second probability of the cell being associated with the second phenotype.
10. The method of any one of embodiments 1-9, wherein the set of probabilities for each cell in the population of cells includes a first probability of the cell being associated with a first biomarker in the plurality of biomarkers and a second probability of the cell being associated with a second biomarker in the plurality of biomarkers.
11. The method of any one of embodiments 1-10, wherein the first subset of cells is identified by generating a reduced dimension representation of a dataset including the set of probabilities associated with each cell in the population of cells.
12. The method of embodiment 11, wherein the reduced dimension representation of the dataset is generated by applying t-distributed stochastic neighbor embedding (t-SNE), uniform manifold approximation and projection (UMAP), principal component analysis (PCA), linear discriminant analysis (LDA), and/or a machine learning model.
13. The method of embodiment 11, wherein the reduced dimension representation of the dataset includes a plurality of cell clusters, and wherein each cell cluster of the plurality of cell clusters includes one or more cells having a same or similar phenotype.
14. The method of embodiment 11, wherein the first subset of cells includes one or more cell clusters from the plurality of cell clusters.
15. The method of any one of embodiments 1-14, wherein the biomarker identification model and/or the first phenotype identification model comprise a gradient boosted decision tree, a random forest, a naïve Bayes classifier, a neural network, a k-means clustering model, or a logistic regression model.
16. The method of any one of embodiments 1-15, further comprising:
training the biomarker identification model to determine, based at least on the plurality of features extracted from the first image, whether one or more cells depicted in the first image is associated with each biomarker in the plurality of biomarkers.
17. The method of embodiment 16, further comprising:
generating, for each cell in the population of cells, an annotated training sample comprising the plurality of features associated the cell and a ground truth label corresponding to each biomarker exhibited by the cell; and
training, based at least on a plurality of annotated training samples, the biomarker identification model.
18. The method of any one of embodiments 1-17, further comprising:
generating, for each cell in the first subset of cells, an annotated training sample comprising the first feature set and a ground truth label corresponding to the first phenotype of the cell; and
training, based at least on a plurality of annotated training samples, the first phenotype identification model.
19. The method of any one of embodiments 1-18, wherein the population of cells is a part of a biological sample or a derivation of the biological sample.
20. The method of any one of embodiments 1-19, wherein the population of cells comprises a tissue fragment and/or a bodily fluid.
21. The method of any one of embodiments 1-20, wherein the first image comprises at least a portion of a whole slide image.
22. The method of any one of embodiments 1-21, wherein the plurality of biomarkers include Pax5, CD68, CD3, CD8, Foxp3, CD335, and/or Ki67.
23. The method of any one of embodiments 1-22, wherein the first phenotype is a transient cell state exhibited by the first subset of cells.
24. The method of any one of embodiments 1-23, wherein the first phenotype is tumor cell, macrophage, regulatory T-cell, CD8-positive T-cell, B-cell, or natural killer (NK) cell.
25. The method of any one of embodiments 1-24, wherein the first image is a whole slide image.
26 The method of any one of embodiments 1-25, wherein the first image is a hematoxylin and eosin (H&E) stained image or a multiplex immunofluorescence (MxIF) stained image.
27. A system, comprising:
at least one data processor; and
at least one memory storing instructions, which when executed by the at least one data processor, result in operations comprising the method of any one of embodiments 1-26.
28. A non-transitory computer readable medium storing instructions, which when executed by at least one data processor, result in operations comprising the method of any one of embodiments 1-26.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

What is claimed is:

1. A computer-implemented method, comprising:
extracting, from an image depicting a population of cells, a plurality of features for each cell in the population of cells;
applying a biomarker identification model to determine, based at least on the plurality of features associated with each cell in the population of cells, whether the cell is associated with, positive for, or negative for a plurality of biomarkers;
determining, based at least on an output of the biomarker identification model, a set of probabilities for each cell in the population of cells, and the set of probabilities including, for each biomarker in the plurality of biomarkers, a probability of a corresponding cell being associated with the biomarker;
identifying, based at least on the set of probabilities associated with each cell in the population of cells, a subset of cells exhibiting a phenotype; and
identifying a feature set associated with the subset of cells as being indicative of a probability of a cell being associated with the phenotype.

2. The method of claim 1, wherein the plurality of features include one or more geometric features, statistical features, and textural features.

3. The method of claim 1, wherein the plurality of features are collected over a plurality of channels, and wherein each channel of the plurality of channels corresponds to (i) an emission wavelength of a fluorescent dye applied to the image, (ii) a metal ion collected by a mass cytometer, (iii) a nucleotide sequence identified by barcode hybridization, or (iv) a nucleotide sequence identified by sequencing.

4. The method of claim 1, wherein each biomarker in the plurality of biomarkers corresponds to a protein of interest or an antigen comprising one or more carbohydrates, lipids, or nucleotides.

5. The method of claim 1, wherein each biomarker in the plurality of biomarkers corresponds to a protein expressed by the population of cells.

6. The method of claim 1, further comprising:
training a phenotype identification model to determine, based on the feature set associated with the subset of cells, the probability of the cell being associated with the phenotype.

7. The method of claim 6, further comprising:
applying the phenotype identification model to determine, based on the feature set extracted from an additional image, a probability of one or more cells depicted in the additional image being associated with the phenotype.

8. The method of claim 7, further comprising:
determining, based at least on the probability of the one or more cells depicted in the additional image being associated with the phenotype, a disease diagnosis, a disease progression, a disease burden, and/or a treatment response.

9. The method of claim 6, further comprising:
identifying, based at least on the set of probabilities associated with each cell in the population of cells, an additional subset of cells exhibiting an additional phenotype; and
training an additional phenotype identification model to determine, based on an additional feature set associated with the additional subset of cells, an additional probability of the cell being associated with the additional phenotype.

10. The method of claim 1, wherein the set of probabilities for each cell in the population of cells includes a probability of the cell being associated with a biomarker in the plurality of biomarkers and an additional probability of the cell being associated with an additional biomarker in the plurality of biomarkers.

11. The method of claim 1, wherein the subset of cells is identified by generating a reduced dimension representation of a dataset including the set of probabilities associated with each cell in the population of cells.

12. The method of claim 1, further comprising:
training the biomarker identification model to determine, based at least on the plurality of features extracted from the image, whether one or more cells depicted in the image is associated with each biomarker in the plurality of biomarkers.

13. The method of claim 12, further comprising:
generating, for each cell in the population of cells, an annotated training sample comprising the plurality of features associated with the cell and a ground truth label corresponding to each biomarker exhibited by the cell; and
training, based at least on a plurality of annotated training samples, the biomarker identification model.

14. The method of claim 1, further comprising:
generating, for each cell in the subset of cells, an annotated training sample comprising the feature set and a ground truth label corresponding to the phenotype of the cell; and
training, based at least on a plurality of annotated training samples, the phenotype identification model.

15. The method of claim 1, wherein the population of cells is a part of a biological sample, a derivation of the biological sample, a tissue fragment and/or a bodily fluid.

16. The method of claim 1, wherein the image comprises at least a portion of a whole slide image.

17. The method of claim 1, wherein the plurality of biomarkers include Pax5, CD68, CD3, CD8, Foxp3, CD335, and/or Ki67.

18. The method of claim 1, wherein the phenotype is a transient cell state exhibited by the subset of cells and/or the phenotype is tumor cell, macrophage, regulatory T-cell, CD8-positive T-cell, B-cell, or natural killer (NK) cell.

19. The method of claim 1, wherein the image is a hematoxylin and eosin (H&E) stained image or a multiplex immunofluorescence (MxIF) stained image.

20. A system, comprising:
at least one data processor; and
at least one memory storing instructions, which when executed by the at least one data processor, result in operations comprising:
extracting, from an image depicting a population of cells, a plurality of features for each cell in the population of cells;
applying a biomarker identification model to determine, based at least on the plurality of features associated with each cell in the population of cells, whether the cell is associated with, positive for, or negative for a plurality of biomarkers;
determining, based at least on an output of the biomarker identification model, a set of probabilities for each cell in the population of cells, and the set of probabilities including, for each biomarker in the plurality of biomarkers, a probability of a corresponding cell being associated with the biomarker;
identifying, based at least on the set of probabilities associated with each cell in the population of cells, a subset of cells exhibiting a phenotype; and
identifying a feature set associated with the subset of cells as being indicative of a probability of a cell being associated with the phenotype.

21. A non-transitory computer readable medium storing instructions, which when executed by at least one data processor, result in operations comprising:
extracting, from an image depicting a population of cells, a plurality of features for each cell in the population of cells;
applying a biomarker identification model to determine, based at least on the plurality of features associated with each cell in the population of cells, whether the cell is associated with, positive for, or negative for a plurality of biomarkers;
determining, based at least on an output of the biomarker identification model, a set of probabilities for each cell in the population of cells, and the set of probabilities including, for each biomarker in the plurality of biomarkers, a probability of a corresponding cell being associated with the biomarker;
identifying, based at least on the set of probabilities associated with each cell in the population of cells, a subset of cells exhibiting a phenotype; and
identifying a feature set associated with the subset of cells as being indicative of a probability of a cell being associated with the phenotype.
